(19) United States
(12) Patent Application Publication
(10) Pub. No.: US 2016/0353974 A1
(43) Pub. Date: Dec. 8, 2016

(54) ENDOSCOPE

(71) Applicant: OLYMPUS CORPORATION, Tokyo (JP)

(72) Inventor: Taigo KAKEHASHI, Tokyo (JP)

(73) Assignee: OLYMPUS CORPORATION, Tokyo (JP)

(21) Appl. No.: 15/244,529

(22) Filed: Aug. 23, 2016

Related U.S. Application Data
(63) Continuation of application No. PCT/JP2015/056503, filed on Mar. 5, 2015.

(30) Foreign Application Priority Data
Apr. 21, 2014 (JP) 2014-087563

(51) Int. Cl. A61B 1/005; G02B 23/24

(52) U.S. Cl. CPC A61B 1/0052; A61B 1/0055; G02B 23/2476

(57) ABSTRACT

An endoscope includes: a functional portion having a function of pressing by rotating around a rotation shaft; a link mechanism attached to the rotation shaft and having a through hole; a shaft member disposed in the through hole and causing the link mechanism to rotate around the rotation shaft; and a slide member fixed to the shaft member and being moved together with the shaft member; and a restricting member configured to restrict the slide member.

### CROSS REFERENCE TO RELATED APPLICATION

This application is a continuation application of PCT/JP2015/056503 filed on Mar. 5, 2015 and claims benefit of Japanese Application No. 2014-087563 filed in Japan on Apr. 21, 2014, the entire contents of which are incorporated herein by this reference.

### BACKGROUND OF THE INVENTION

### 1. Field of the Invention

The present invention relates to an endoscope provided with a slide member configured to be slid accompanying a lever operation.

### 2. Description of the Related Art

Recently, insertion apparatuses have been used in medical and industrial fields.

In the medical field, an endoscope, which is one of the insertion apparatuses, is used. The endoscope makes it possible to perform various kinds of treatments by inserting a treatment instrument into a treatment instrument insertion channel provided in the endoscope in addition to in-vivo observation, by an elongated insertion portion being inserted into a body.

Among endoscopes, an endoscope that is provided with a bending portion configured to be bendable, at a distal end of the insertion portion, an endoscope that is provided with a treatment instrument raising base for guiding a treatment instrument inserted into the insertion portion in a predetermined direction, in a distal end portion of the insertion portion, an endoscope that is provided with an image pickup unit capable of any of changing observation magnification, focusing and zooming by moving an optical lens of an objective optical system, at the distal end portion, and others are well known.

Japanese Patent Application Laid-Open Publication No. 11-225946 discloses an endoscope system which can be configured inexpensively by causing a wire pulling mechanism configured to move an optical lens of an objective optical system which a first electronic endoscope is provided with and a wire pulling mechanism configured to raise a treatment instrument raising base which a second electronic endoscope is provided with to mutually share at least a part of their components.

A bending portion is configured to bend, for example, in four directions of upward, downward, left and right directions by rotatably coupling a plurality of bending pieces, and performs a bending operation, for example, in the upward direction and an intermediate direction between the upward direction and the left direction by a user operating the wire pulling apparatus to pull or loosen a bending wire.

Recently, various endoscope configurations have been proposed in which a so-called bending length switching mechanism making it possible to switch a bending length of the bending portion is provided.

An insertion portion of an endoscope provided with the bending length switching mechanism is configured with a distal end portion, a bending portion having a first bending portion and a second bending portion, and a flexible tube portion being connected. The bending portion is configured so that a state in which only the first bending portion bends and a state in which the first and second bending portions bend together can be switched.

The endoscope provided with the bending length switching mechanism is provided with a bending wire, a first coil sheath and a second coil sheath in the insertion portion and an operation portion. The bending wire is inserted in the first coil sheath, and the first coil sheath is inserted in the second coil sheath.

A distal end and proximal end of the bending wire extend from respective end surfaces of the first coil sheath. The distal end of the bending wire is fixed to a distal end bending piece constituting a first bending portion, and the proximal end is connected to a wire pulling apparatus provided on the operation portion.

On the other hand, a distal end and a proximal end of the first sheath extend from respective end surfaces of the second coil sheath. The distal end of the first coil sheath is fixed between the first and second bending portions, and, as for the proximal end, fixed/non-fixed can be switched in the operation portion.

A distal end of the second coil sheath is fixed to a distal end of the flexible tube, and a proximal end is fixed to a predetermined part in the operation portion.

In the endoscope configured as described above, the bending portion performs a bending operation by a rotation operation of a bending knob to cause the wire pulling apparatus to operate and cause the bending wire to be pulled or loosened. If, in a bending operation state, a proximal end of the first coil sheath is in a fixed state, only the first bending portion of the bending portion at a more distal end side than the first coil sheath bends. In comparison, if the proximal end of the first coil sheath is in a non-fixed state, the first and second bending portions of the bending portion at a more distal end side than the second coil sheath bend together.

Switching of the fixed/non-fixed of the proximal end of the first coil sheath is performed by performing a rotation operation of a switching operation lever provided on the operation portion.

Japanese Patent No. 5253689 shows an insertion apparatus provided with a fixing mechanism configured to, when it is desired to keep an operating portion of an insertion portion in a desired operation state, prevent the state from being easily released, by an external force acting on the operating portion. In a third embodiment (see FIGS. 22 to 24 and the like), a switching mechanism configured to switch a first bending portion between a movable state and a fixed state by operating a switching operation lever is shown.

In the switching mechanism of Japanese Patent No. 5253689, a moving unit is caused to operate accompanying an operation of the switching operation lever, and a slide member starts to move along a longitudinal axis. After that, two links constituting a link unit start to rotate relative to a coupling portion, together with the moving slide member. Accompanying the rotation, a brake pad attached to a brake caliper coupled with each link comes into contact with a moving body attached to a bending wire and generates a restricting force. Thereby, the first bending portion is fixed and held.

The switching mechanism configured to generate a restricting force as described above is effective as a coil sheath switching mechanism configured to switch the proximal end of the first coil sheath between the fixed state and the non-fixed state.

### SUMMARY OF THE INVENTION

An endoscope of an aspect of the invention includes: a functional portion having a function of, by rotating around a rotation shaft formed in a first direction, pressing in a second direction from a pressing portion formed on an outer circumferential surface; a link mechanism attached to the rotation shaft, rotatable around the rotation shaft, and having a through hole formed in the first direction; a shaft member disposed in the through hole and capable of moving in a cross direction crossing the first and second directions by causing the link mechanism to rotate around the rotation shaft; a slide member fixed to the shaft member, having a longitudinal axis in the cross direction, and being moved in the cross direction together with the shaft member by an operation force for causing the functional portion to function being inputted; and a restricting member configured to restrict the slide member so as not to move in a direction different from the cross direction.

### BRIEF DESCRIPTION OF THE DRAWINGS

FIG. 1 is a diagram illustrating an endoscope which is an insertion apparatus provided with a functional portion according to the present embodiment;

FIG. 2 is a diagram illustrating a bending mechanism of a bending portion which is a function part the endoscope is provided with;

FIG. 3 is a cross-sectional view of a Y3-Y3 line of FIG. 2;

FIG. 4 is a cross-sectional view of a Y4-Y4 line of FIG. 2;

FIGS. 5 to 15 are diagrams illustrating a bending mechanism switching portion provided in an operation portion, and FIG. 5 is a diagram illustrating a configuration when inner coils provided in the bending mechanism switching portion are in a non-fixed state;

FIG. 6 is a cross-sectional view of a Y6-Y6 line of FIG. 5;

FIG. 7 shows the bending mechanism switching portion on a first end side of a slide member when a link mechanism and a shaft member are removed from the diagram of FIG. 5;

FIG. 8 shows the bending mechanism switching portion on the first end side of the slide member when the link mechanism and a cover member are removed from the diagram of FIG. 5;

FIG. 9 is a cross-sectional view of a Y9-Y9 line of FIG. 8;

FIG. 10 is a diagram illustrating the bending mechanism switching portion when the inner coils are in a fixed state;

FIG. 11 shows the bending mechanism switching portion on the first end side of the slide member when the link mechanism and the shaft member are removed from the diagram of FIG. 10;

FIG. 12 shows the bending mechanism switching portion on the first end side of the slide member when the link mechanism and the cover member are removed from the diagram of FIG. 10;

FIG. 13 is a cross-sectional view of a Y13-Y13 line of FIG. 12;

FIG. 14 is a diagram illustrating another configuration example of the bending mechanism switching portion;

FIG. 15 is a diagram illustrating a different configuration example of the bending mechanism switching portion and is a diagram showing the inner coil non-fixed state;

FIG. 16 is a cross-sectional view of a Y16-Y16 line of FIG. 15; and

FIG. 17 is a diagram showing the inner coil fixed state of the bending mechanism switching portion of FIG. 15.

### DETAILED DESCRIPTION OF THE PREFERRED EMBODIMENT(S)

An embodiment of the present invention will be described below with reference to drawings.

Note that each drawing used in description below is schematic, and a dimensional relationship, reduced scale and the like of each member may be shown different for each component so that each component is recognizably shown on the drawing. Therefore, the present invention is not limited to shown forms, such as the number of components, shapes of the components, a ratio of sizes of the components, and relative positional relationship among the respective components shown in the drawings.

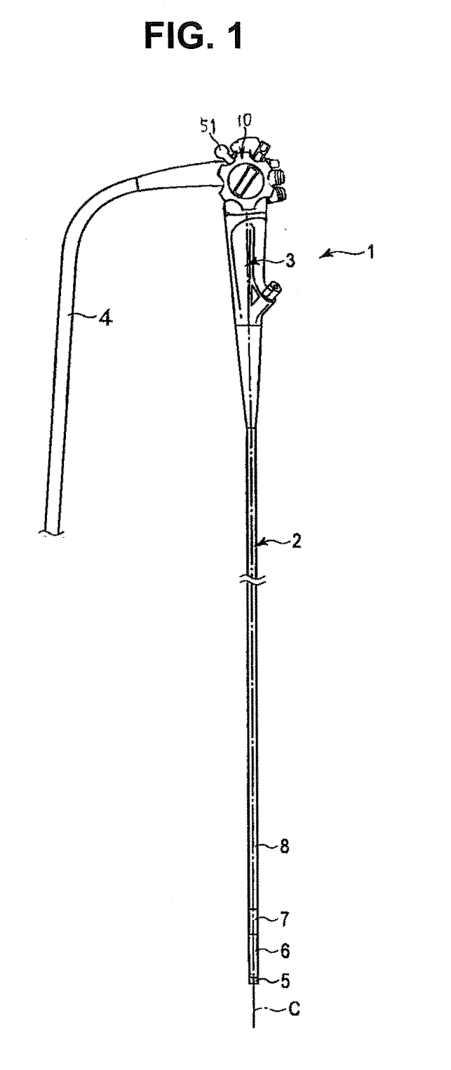

As shown in FIG. 1, an endoscope 1, which is one of insertion apparatuses, is configured having an insertion portion 2 configured to be inserted into a subject, an operation portion 3 and a universal cord 4. The insertion portion 2 is an elongated tube portion having flexibility and is extended along a longitudinal axis C. The operation portion 3 is arranged being connected to an end portion on an opposite side of an insertion direction of the insertion portion 2.

The operation portion 3 is provided mainly with a bending operation knob 10, which is a bending operation device, and a switching lever 51.

The insertion portion 2 is elongatedly configured having a distal end portion 5, a first bending portion 6, a second bending portion 7 and a flexible tube portion 8 being connected in that order from a distal end side.

On a distal end surface of the distal end portion 5, an illuminating window (not shown) constituting an illumination optical system configured to illuminate a subject portion, an observation window (not shown) constituting an image pickup optical system configured to pick up an image of an illuminated subject part, a nozzle (not shown) configured to eject fluid for removing body fluid and the like adhering to the observation window or the illuminating window, a distal end opening (not shown) of a treatment instrument insertion channel (not shown) and the like, are provided.

In the present embodiment, a bending portion of the insertion portion 2 is configured including the first bending portion 6 and the second bending portion 7. The bending portion is configured to bend in upward and downward directions accompanying an operation of the bending operation knob 10.

The switching lever 51 switches between a short bending state in which only the first bending portion 6 constituting the bending portion bends and a long bending state in which the first and second bending portions 6 and 7 constituting the bending portion bend together, accompanying an operation of the bending operation knob 10.

That is, a user can selectively switch the bending portion between the long bending state and the short bending state by operating the switching lever 51.

Note that, though bending directions of the bending portion are the two directions of the upward and downward directions in the above description, the bending directions of the bending portion are not limited to the two directions of the upward and downward directions, and a configuration is also possible in which the bending portion bends in four directions of upward, downward, left and right directions.

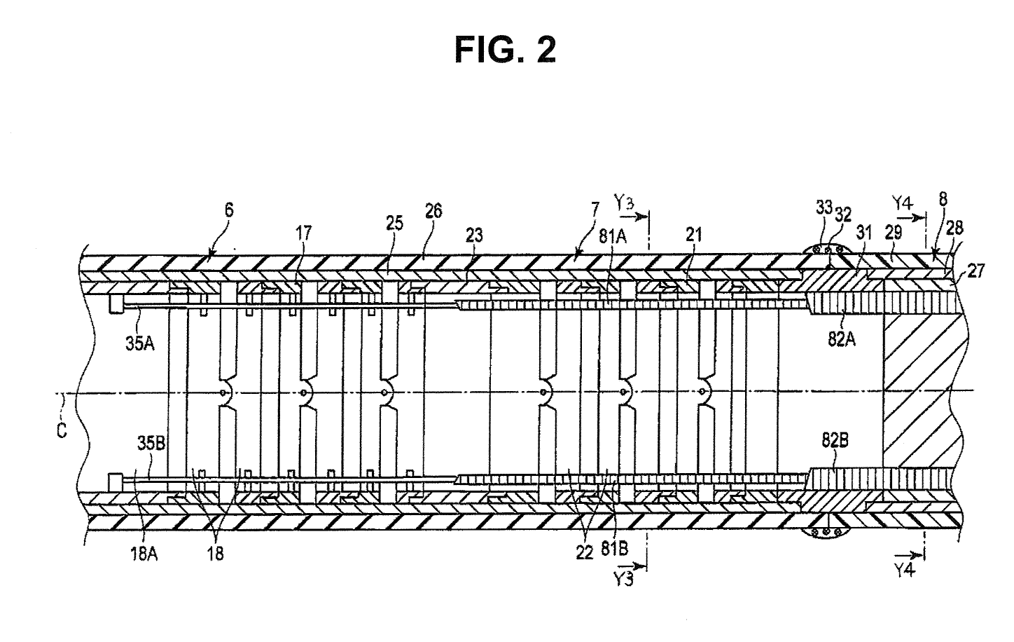

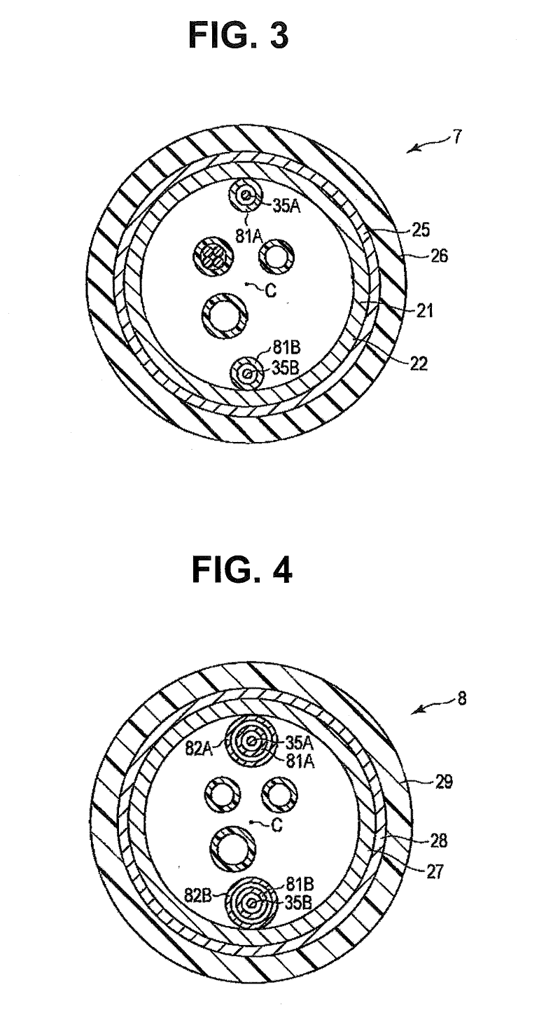

Configurations of the first bending portion 6, the second bending portion 7 and the flexible tube portion 8 will be described with reference to FIGS. 2 to 4.

The first bending portion 6 is provided with a first bending portion group 17. The first bending portion group 17 is configured with a plurality of first bending pieces 18 arranged being connected along the longitudinal axis C. Each of the first bending pieces 18 is mutually rotatably coupled with an adjoining first bending piece 18.

The second bending portion 7 is provided with a second bending portion group 21. The second bending portion group 21 is configured with a plurality of second bending pieces 22 arranged being connected along the longitudinal axis C. Each of the second bending pieces 22 is mutually rotatably coupled with an adjoining second bending piece 22.

The first bending portion group 17 and the second bending portion group 21 are coupled with a first connection pipe sleeve 23. A first netlike tube (a first braid) 25 made of metal is provided on an outer circumferential direction side of the first bending portion group 17 and the second bending portion group 21. A first outer cover 26 made of rubber is covered on an outer circumferential direction side of the first netlike tube 25.

The flexible tube portion 8 is provided with a spiral tube (a flex) 27 made of metal. On an outer circumferential direction side of the spiral tube 27, a second netlike tube (a second braid) 28 made of metal is provided. A second outer cover 29 made of resin is covered on an outer circumferential direction side of the second netlike tube 28.

The first netlike tube 25 of the second bending portion group 21, the spiral tube 27 and the second netlike tube 28 are coupled via a second connection pipe sleeve 31. A thread 32 is wound around an outer circumferential surface of a proximal end portion of the first outer cover 26 and an outer circumferential surface of a distal end portion of the second outer cover 29, and a bobbin winding/bonding portion to which adhesive 33 is applied is provided around the thread 32.

As shown in FIGS. 2 to 4, distal ends of bending operation wires 35A and 35B are fixed to a first bending piece 18 located on a most distal end direction side among the first bending pieces 18. Each of the bending operation wires 35A and 35B is extended along the longitudinal axis C inside the insertion portion 2. The respective bending operation wires 35A and 35B are disposed at positions substantially facing each other with the longitudinal axis C between them.

Inside the insertion portion 2, two first coil pipes (hereinafter referred to as inner coils) 81A and 81B are extended along the longitudinal axis C. The corresponding bending operation wires 35A and 35B are inserted in the respective inner coils 81A and 81b. The respective inner coils 81A and 81B are disposed at positions substantially facing each other with the longitudinal axis C between them. Distal ends of the respective inner coils 81A and 81B are fixed to the first connection pipe sleeve 23.

Further, inside the insertion portion 2, two second coil pipes (hereinafter referred to as outer coils) 82A and 82B are extended along the longitudinal axis C as shown in FIGS. 2 and 4. The corresponding inner coils 81A and 81B are inserted in the respective outer coils 82A and 82B. The respective outer coils 82A and 82B are disposed at positions substantially facing each other with the longitudinal axis C between them.

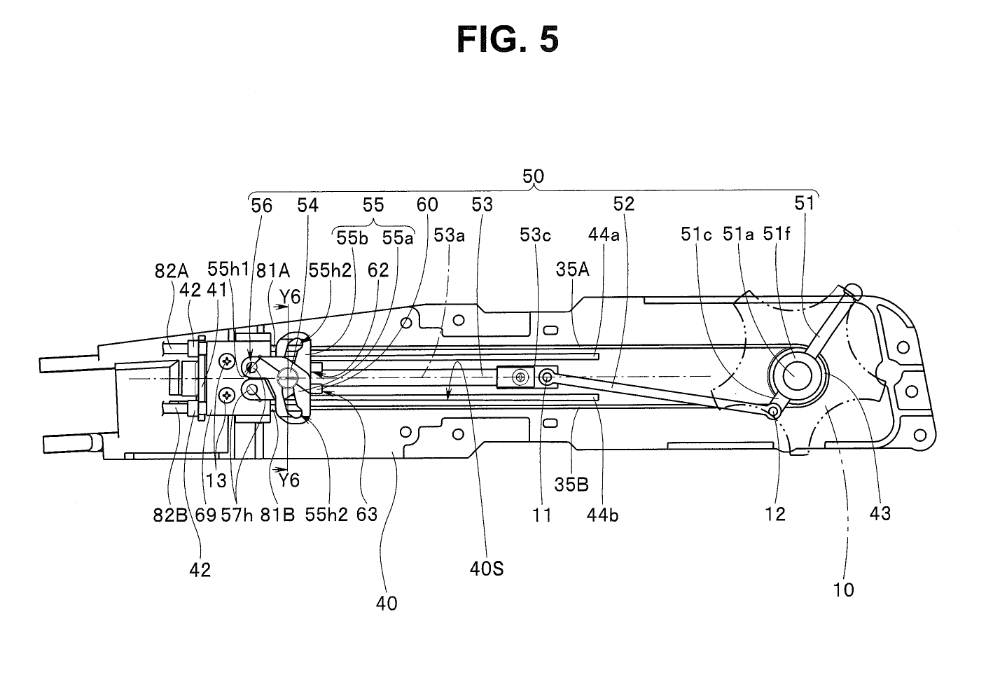

Distal ends of the respective outer coils 82A and 82B are fixed to the second connection pipe sleeve 31. Proximal ends of the respective outer coils 82A and 82B are fixed to an outer coil fastening plate 41 arranged standing on a bottom plate 40, which is a main plate shown in FIG. 5, the outer coil fastening plate 41 being fixedly arranged in the operation portion 3. Reference numeral 42 denotes an outer coil fastening member.

Proximal ends of the respective inner coils 81A and 81B are extended from the proximal ends of the respective outer coils 82A and 82B and slidably disposed at predetermined positions on the bottom plate 40. Further, proximal ends of the respective bending operation wires 35A and 35B are extended from the proximal ends of the inner coils 81A and 81B and fixed, for example, to a pulley 43.

Reference numeral 50 shown in FIG. 5 denotes a bending mechanism switching portion, which is provided on the bottom plate 40 arranged in internal space of an exterior member constituting the operation portion 3. A plate longitudinal axis of the bottom plate 40 is disposed substantially parallel to the longitudinal axis C.

The bending mechanism switching portion 50 is configured mainly having the switching lever 51, a lever operation force transmitting member (hereinafter shortly referred to as a transmitting member) 52, a slide member 53, a shaft member 54, a link mechanism portion 55, a fixing function portion 56, and a restricting member 60.

The link mechanism portion 55 has a first link member 55a and a second link member 55b. The fixing function portion 56 has a cam body 57 and a brake member 58.

The switching lever 51 is configured with a lever shaft 51a and a lever flange 51f projecting from an outer circumferential surface of the lever shaft 51a being integrally fixed. The lever flange 51f is provided with a coupling projection portion 51c in addition to the switching lever 51. The coupling projection portion 51c is provided at a predetermined position, for example on an opposite side of the switching lever 51 with the lever shaft 51a between the coupling projection portion 51c and the switching lever 51.

The transmitting member 52 is, for example, a plate-shaped member. A coupling hole is formed on each of a distal end portion, which is a first end of the transmitting member, and a proximal end portion, which is a second end.

A first coupling pin 11 is arranged in the first coupling hole, and a second coupling pin 12 is arranged in the second coupling hole.

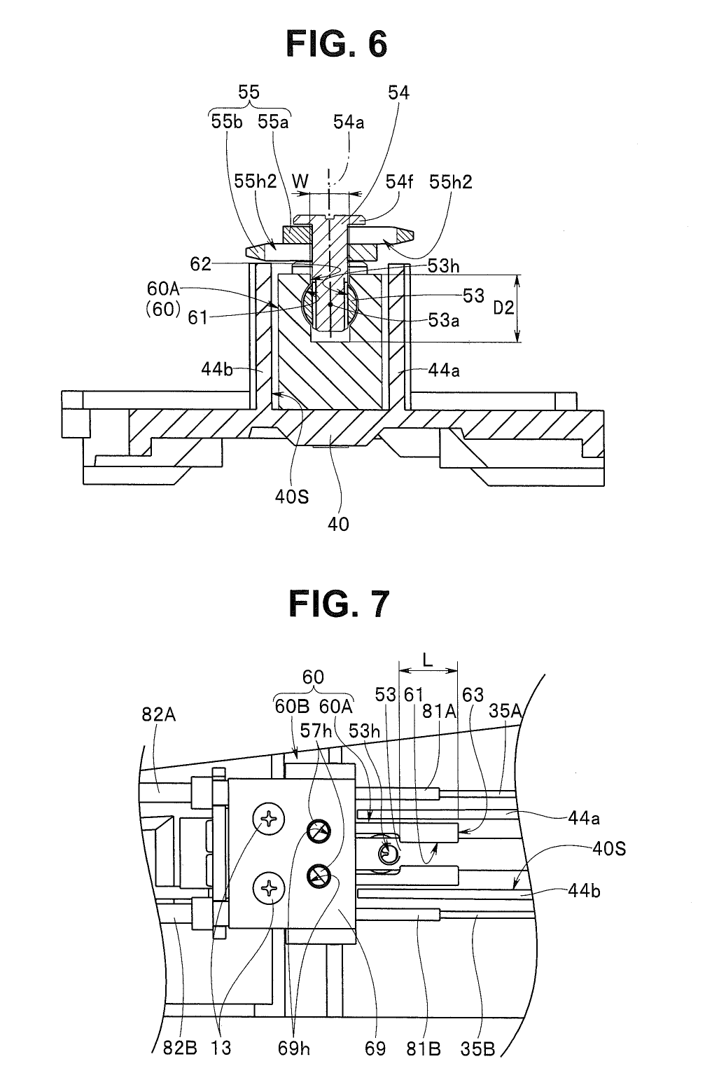

The slide member 53 is provided with a distal end portion, which is a first end, and a proximal end portion, which is a second end, and has a longitudinal axis 53a extending from the distal end portion to the proximal end portion. As shown in FIG. 6, a shape of a cross section of the slide member 53 is a circle.

As shown in FIGS. 5 and 6, a distal end portion of the slide member 53 is slidably disposed in a restricting hole 61 of the restricting member 60, and a remaining part is slidably disposed in slide space 40S provided for the bottom plate 40.

The slide space 40S is a recess groove configured with a surface of the bottom plate 40 and inner wall surfaces of a first projection portion 44a and a second projection portion 44b projecting from the surface of the bottom plate 40 and extended along a plate longitudinal axis.

Reference numeral 62 denotes a shaft member moving groove on which a shaft body of the shaft member 54 is slidably disposed.

A coupling cover 53c which is, for example, in a cylindrical shape is fixed to a proximal end portion of the slide member 53. A distal end portion of the transmitting member 52 is rotatably coupled with the coupling cover 53c via the first coupling pin 11. An axis of the first coupling pin 11 is provided substantially orthogonal to the longitudinal axis 53a of the slide member 53 so that the axis and the longitudinal axis 53a cross.

On the other hand, the second coupling pin 12 rotatably couples a proximal end portion of the transmitting member 52 with the lever flange 51f. An axis of the second coupling pin 12 is provided on one surface of the coupling projection portion 51c so as to stand substantially straight.

Note that the proximal end portion of the slide member 53 and the distal end portion of the transmitting member 52 may be rotatably coupled by the first coupling pin 11.

As described above, according to the configuration in which the coupling cover 53c, the transmitting member 52, and the lever flange 51f on which the switching lever 51 and the coupling projection portion 51c are provided are coupled via the first coupling pin 11 and the second coupling pin 12, a rotational motion of the lever flange 51f is converted to a linear motion of the slide member 53.

That is, the proximal end portion of the slide member 53 is an input portion to which an operation force generated when the switching lever 51 is operated is inputted.

As shown in FIG. 5, the shaft member 54 is provided at a position separated from the first coupling pin 11 to a first end side by a predetermined distance. As shown in FIG. 6, the shaft member 54 is integrally fixed to the slide member 53 through shaft member arrangement through holes 55h2 of the first link member 55a and the second link member 55b constituting the link mechanism portion 55.

In the present embodiment, the shaft member 54 has a male screw at a distal end portion of the shaft body and a flange 54f at a proximal end portion of the shaft body. The flange 54f restricts the link members 55a and 55b so as not to move in a direction of an axis 54a of the shaft member 54.

The male screw of the shaft member 54 is screwed with a female screw provided in a through hole 53h formed in the slide member 53. The shaft member 54 is provided so that the axis 54a and the longitudinal axis 53a of the slide member 53 cross.

The axis 54a of the shaft member 54 fixed so that the axis 54a and the longitudinal axis 53a of the slide member 53 cross and the axis of the first coupling pin 11 project in a same direction from a same outer circumferential position, for example, when seen from a front, from one end surface side of the slide member 53.

The first and second link members 55a and 55b constituting the link mechanism portion 55 shown in FIGS. 5 and 6 are plate members and are formed in a predetermined bending shape. By appropriately adjusting lengths of the first and second link member 55a and 55b, a direction of a force acting in a direction of the longitudinal axis 53a of the slide member 53 can be changed.

The first and second link members 55a and 55b are provided with, for example, a cam fixing through hole 55h1 which is, for example, a round hole, and the shaft member arrangement through hole 55h2 which is a predetermined cam-shaped long hole extending from a central part to a proximal end.

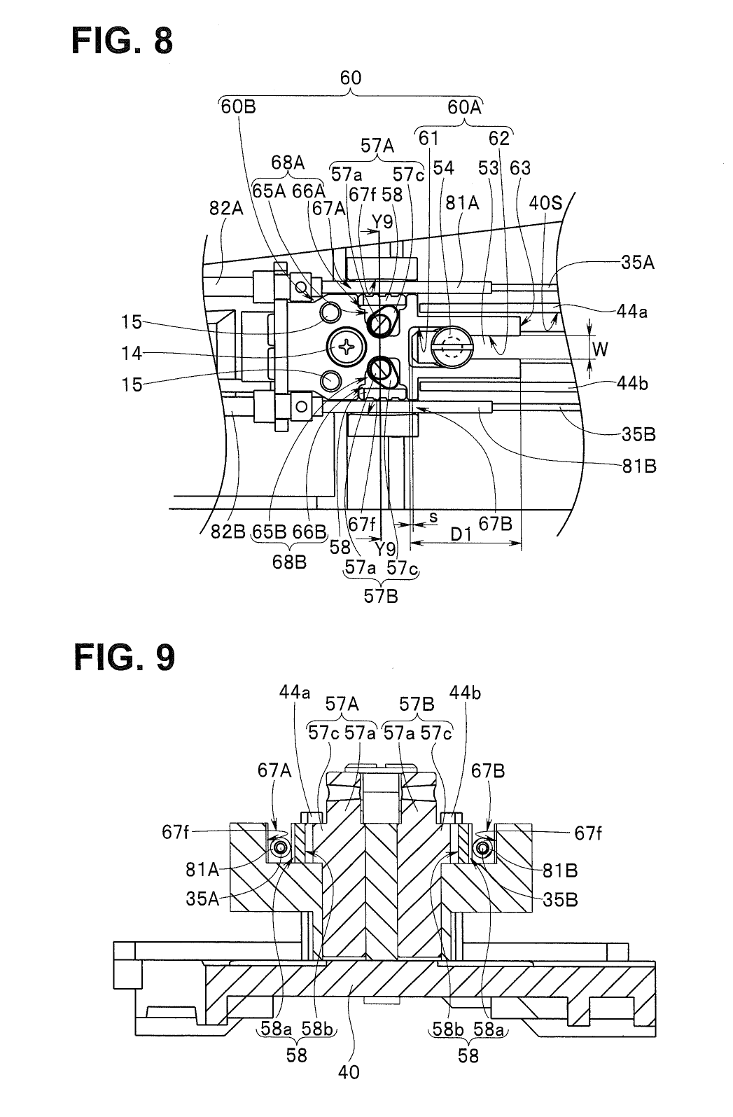

A head portion 57h of a first cam body (see reference numeral 57A in FIGS. 8 and 9) constituting the fixing function portion 56 is integrally fixed in the cam fixing through hole 55h1 of the first link member 55a, and a head portion 57h of a second cam body (see reference numeral 57B in FIGS. 8 and 9) is integrally fixed in the cam fixing through hole 55h1 of the second link member 55b.

On the other hand, the shaft body of the shaft member 54 is disposed in the shaft member arrangement through holes 55h2 of the first and second link members 55a and 55b.

Reference numeral 69 denotes a cover member. The cover member 69 is integrally fixed to the restricting member 60 by a fixing screw 13.

The fixing function portion 56 and the restricting member 60 will be described with reference to FIGS. 5 to 9.

As shown in FIGS. 7 and 8, the restricting member 60 is provided with a restricting portion body 60A and a functional portion constituting portion 60B. The restricting member 60 is integrally fixed at a predetermined position of the bottom plate 40 with a fixing screw 14.

As shown in FIGS. 6 to 8, the restricting portion body 60A is provided with the restricting hole 61 and the shaft member moving groove 62. A diameter dimension of the restricting hole 61 is set to correspond to a predetermined fitting state so that the slide member 53 smoothly moves back and forth without backlash.

A depth dimension D1 of the restricting hole 61 is set to a predetermined dimension from an end surface 63 so that a clearance s is provided in order to prevent a distal end surface of the slide member 53 from coming into contact with a bottom surface of the restricting hole 61 when the inner coils are in the non-fixed state.

On the other hand, a width dimension W of the shaft member moving groove 62 is set wider than an outer diameter dimension of the shaft body of the shaft member 54 in advance. Further, a length dimension L of the shaft member moving groove 62 is set to a predetermined dimension from the end surface 63.

In the inner coil non-fixed state, the shaft body of the shaft member 54 is disposed on a more distal end side than a distal end side end of the shaft member moving groove 62. The shaft body of the shaft member 54 is disposed in the shaft member moving groove 62 in the inner coil non-fixed state. That is, almost simultaneously when the inner coil non-fixed state is switched to the inner coil fixed state, and the slide member 53 is moved to a proximal end side in an axial direction, the shaft body of the shaft member 54 is disposed in the shaft member moving groove 62.

Further, a depth dimension D2 of the shaft member moving groove 62 is set to a predetermined dimension from a link member disposing surface side. A bottom surface of the shaft member moving groove 62 is provided at a position beyond the restricting hole 61. According to the configuration, when screwing the shaft member 54 with the slide member 53, it is possible to cause a screw distal end to project from the restricting hole 61 and adjust a separation distance between one surface of the flange 54f and a link member disposing surface 64 while securing the screwed state.

As shown in FIGS. 7 to 9, the functional portion constituting portion 60B is provided with cam body arranging portions 65A and 65B, brake member arranging portions 66A and 66B, and inner coil arranging portions 67A and 67B. Reference numeral 15 denotes a female screw hole, which is a female screw portion having an opening on a cover fitting surface side and with which the fixing screw 13 is screwed.

As shown in FIG. 9, the functional portion constituting portion 60B is provided with the inner coil arranging portions 67A and 67B where proximal end sides of the inner coils 81A and 81B are arranged. As shown in FIGS. 8 and 9, the inner coil arranging portions 67A and 67B are recess grooves each of which has an inner coil pressing/holding surface 67f.

As shown in FIGS. 7 to 9, the cam body arranging portion 65A and the brake member arranging portion 66A constitute a functional member arranging portion 68A, and the cam body arranging portion 65B and the brake member arranging portion 66B constitute a functional member arranging portion 68B. The respective functional member arranging portions 68A and 68B are provided facing the inner coil pressing/holding surfaces 67f of the inner coil arranging portions 67A and 67B, respectively. The cam body arranging portions 65A and 65B are recess grooves from a cover fitting surface to a bottom plate fitting surface and are formed so that the cam body 57 is rotatably arranged.

The brake member arranging portions 66A and 66B are recesses provided on an inner coil pressing/holding surfaces 67f side as compared to the cam body arranging portions 65A and 65B, and the brake member arranging portions 66A and 66B are recesses from the cover fitting surface to a vicinity of recess groove bottom surfaces of the inner coil arranging portions 67A and 67B, where the brake member 58 is movably accommodated and disposed toward a direction of the inner coil pressing/holding surfaces 67f.

As a result, each of the inner coils 81A and 81B is arranged between the brake member 58 and the inner coil pressing/holding surfaces 67f.

Note that a width dimension of the recesses to be the brake member arranging portions 66A and 66B is wider than a width dimension of the recess grooves to be the cam body arranging portions 65A and 65B.

The fixing function portion 56 is configured having the cam body 57 and the brake member 58. The cam body 57 has a shaft portion 57a and a cam portion 57c. The shaft portion 57a is in a cylindrical shape, and the cam portion 57c is a projection portion projecting from an outer circumferential surface of the shaft portion 57a and formed in a predetermined shape.

A distal end side end surface of the cam portion 57c is formed in a curved shape, and the cam portion 57c is a pressing portion configured to press the brake member 58.

The brake member 58 has a contact surface 58b with which the cam portion 57c comes into contact, and a pressing surface 58a configured to come into contact with and press outer surfaces of the inner coils 81A and 81B. The contact surface 58b is a plane, and the pressing surface 58a is configured, for example, with projections portions and recess portions being continuously provided.

Description will be made on operation of the endoscope 1 configured as described above.

At time of using the endoscope 1, a surgeon operates the switching lever 51 to select whether the short bending state in which only the first bending portion 6 of the bending portion is caused to bend or the long bending state in which the first bending portion 6 and the second bending portion 7 are caused to bend together is to be set.

In a case of performing observation in the long bending state, the surgeon causes the inner coil 81A to be in the non-fixed state. That is, the inner coil 81A is caused to be in a state of FIGS. 8 and 9. When the surgeon performs an operation for causing the bending operation knob 10 to bend in the upward direction in the long bending state, the bending operation wire 35A is pulled, and the first bending portion 6 of the bending portion is gradually bent. Then, accompanying the bending of the first bending portion 6, a compressive force along an extension direction acts on the inner coil 81A the distal end of which is fixed to the first connection pipe sleeve 23.

When the compressive force becomes larger than a predetermined amount of force accompanying the bending of the first bending portion 6, a proximal end of the inner coil 81A in the non-fixed state cannot resist the compressive force and moves in a direction of the switching lever 51.

In comparison, the outer coil 82A in the flexible tube portion 8 resists the compressive force which acts along an extension direction of the outer coil 82A because a distal end of the outer coil 82A is fixed to the second connection pipe sleeve 31, and a proximal end is fixed to the outer coil fastening plate 41.

As a result, in the bending portion, the first bending portion 6 and the second bending portion 7 constituting the bending portion perform a bending operation together, with the distal end of the outer coil 82A as a starting point.

On the other hand, in a case of performing examination in the short bending state, that is, in a case of causing only the first bending portion 6 of the bending portion to bend to perform examination, the surgeon operates the switching lever 51 and performs an operation for switching the inner coil 81A from the non-fixed state to the fixed state.

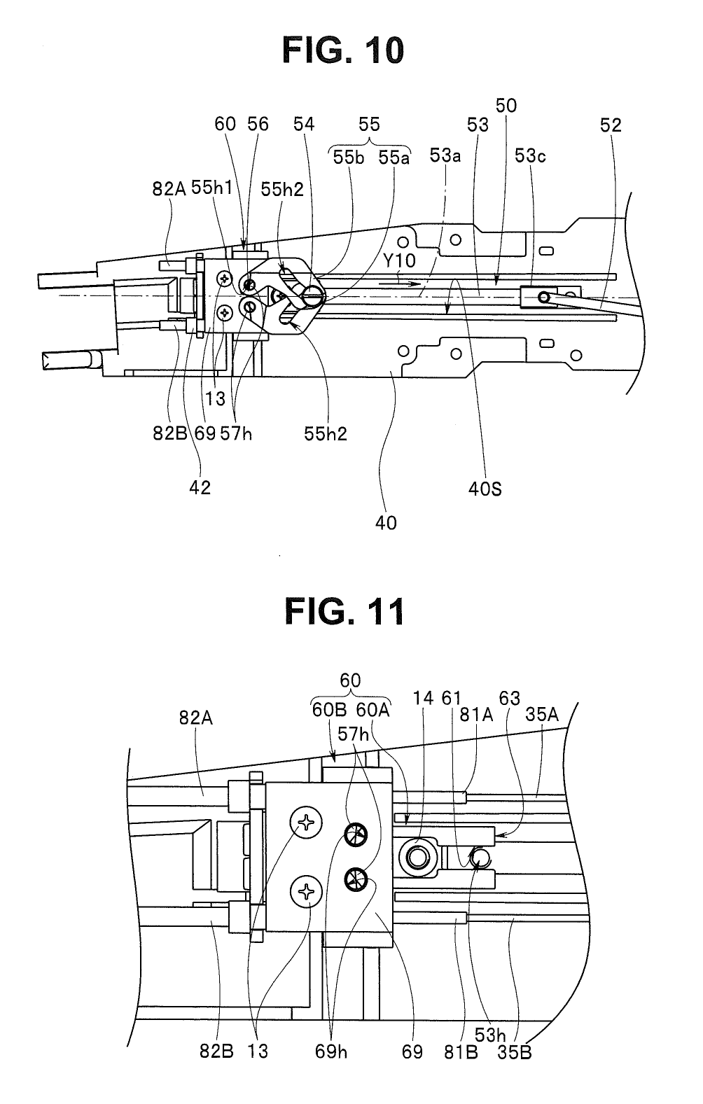

When the switching lever 51 is operated, the lever flange 51f starts rotation by the operation force, and, accompanying the rotation, the transmitting member 52 is moved. Then, accompanying the movement of the transmitting member 52, the operation force is transmitted to the slide member 53, and the slide member 53 is moved in a direction of the lever as shown by an arrow Y10 in FIG. 10.

At this time, the slide member 53 smoothly moves in the restricting hole 61 in the direction of the lever, and the shaft body of the shaft member 54 fixed to a first end of the slide member 53 moves in the same direction in the shaft member moving groove 62.

The moving shaft body of the shaft member 54 is disposed in the shaft member arrangement through holes 55h2 of the first and second link members 55a and 55b. Therefore, the shaft member 54 gradually moves in the shaft member arrangement through holes 55h2 from a central part to a proximal end. At this time, the flange 54f of the shaft member 54 moves while restricting the link members 55a and 55b so as not to move in the direction of the axis 54a of the shaft member 54.

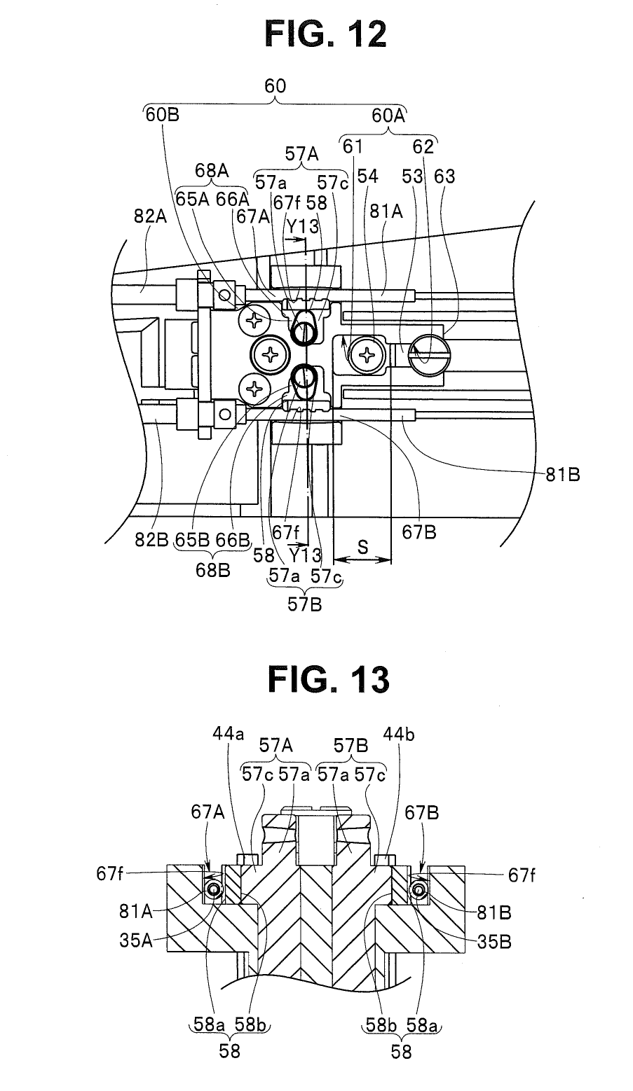

Then, as shown in FIGS. 11 and 12, the slide member 53 completes the movement, and the clearance between the distal end surface of the slide member 53 and the bottom surface of the restricting hole 61 expands to be S.

As a result, as shown in FIGS. 10 to 13, the first and second link member 55a and 55b are rotated, and the shaft portions 57a of the cam bodies 57A and 57B which are arranged in the cam body arranging portions 65A and 65B and the head portions 57h of which are fixed to the cam fixing through hole 55h1 are rotated.

Accompanying the rotation of the shaft portions 57a, the distal end side end surfaces of the cam portions 57c come into contact with the contact surfaces 58b of the brake members 58. After that, the cam portions 57c causes the brake members 58 to move further by the shaft portions 57a being further rotated. As a result, the pressing surfaces 58a of the brake members 58 come into contact with outer surface of proximal end portions of the inner coils 81A and 81B and are in a pressing state. In the pressing state, the proximal end portions of the inner coils 81A and 81B are pressed by the inner coil pressing/holding surfaces 67f and switched from the non-fixed state to the fixed state, and, thereby, the inner coils 81A and 81B are in the short bending state.

When the surgeon operates the bending operation knob 10 to perform, for example, an operation for causing the bending portion to bend in the upward direction in the short bending state, the first bending portion 6 of the bending portion is gradually bent, accompanying the bending operation wire 35A being pulled. Then, accompanying the bending, a compressive force along an extension direction acts on the inner coil 81A the distal end of which is fixed to the first connection pipe sleeve 23. At this time, since a proximal end portion of the inner coil 81A is caused to be in the fixed state by the brake member 58, the inner coil 81A resists the compressive force acting on the inner coil 81A along the extension direction applied to the inner coil 81A.

As a result, only the first bending portion 6 of the bending portion performs a bending operation with the distal end of the inner coil 81A as a starting point.

Thus, the shape of the cross section of the slide member 53 constituting the bending mechanism switching portion 50 is formed in a circular shape, and the distal end portion of the slide member 53 is slidably disposed in the restricting hole 61 of the restricting member 60 in a predetermined fitting state. Further, the shaft body of the shaft member 54 is caused to be disposed on a more distal end side than the distal end side end of the shaft member moving groove 62 in the inner coil non-fixed state and disposed in the shaft member moving groove 62 in the inner coil fixed state to restrict the link members 55a and 55b from moving in the direction of the axis 54a of the shaft member 54 by the flange 54f.

As a result, the slide member is prevented from moving in a cross direction by an operation force at time of operating the lever being transmitted to the slide member. As a result, the operation force is efficiently transmitted to the slide member and the two links which are disposed horizontally relative to the surface of the bottom plate 40, and, therefore, it is possible to perform smooth switching work. Further, by the brake member 58 pressing the inner coils 81A and 81B in the state that the slide member and the two links are disposed horizontally relative to the surface of the bottom plate 40, it is possible to cause the inner coils 81A and 81B to be in the fixed state by a predetermined restricting force.

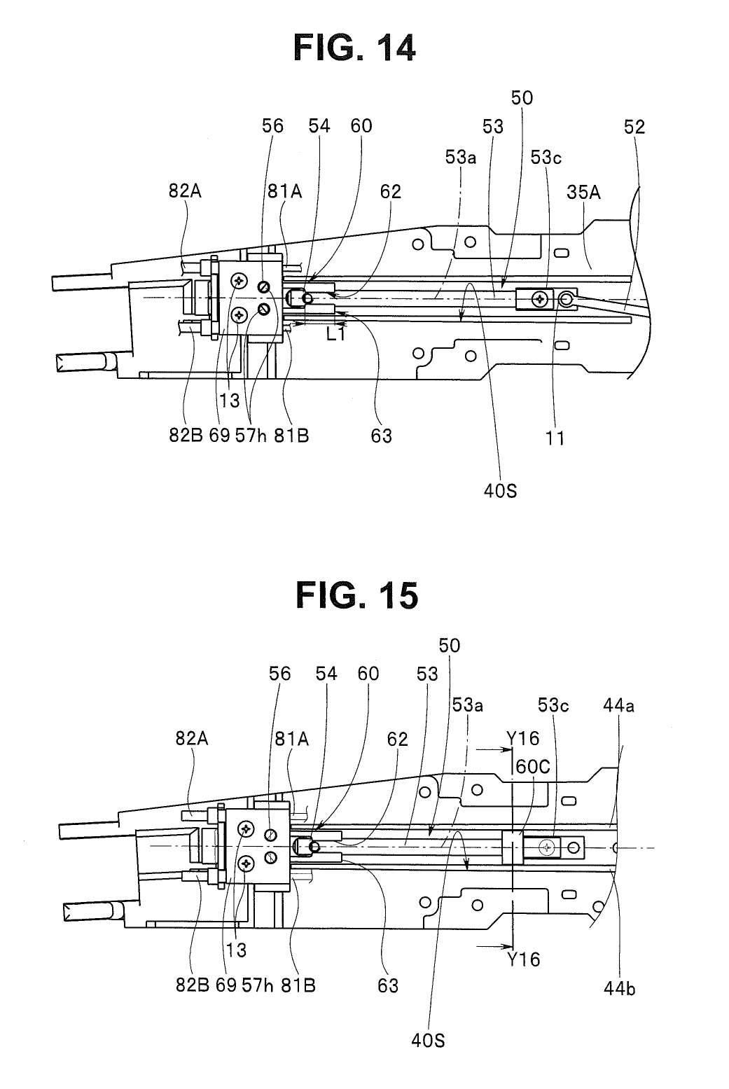

Note that, as shown in FIG. 14, a length dimension L1 of the shaft member moving groove 62 may be specified so that the shaft body of the shaft member 54 is disposed in the shaft member moving groove 62 while the slide member 53 is moved to the proximal end side in the shaft direction, irrespective of whether the inner coil non-fixed state or the inner coil fixed state. Thereby, the slide member is more certainly prevented from moving in the cross direction while an operation force is transmitted, and it is possible to obtain a smooth switching operation and a stable restricting force.

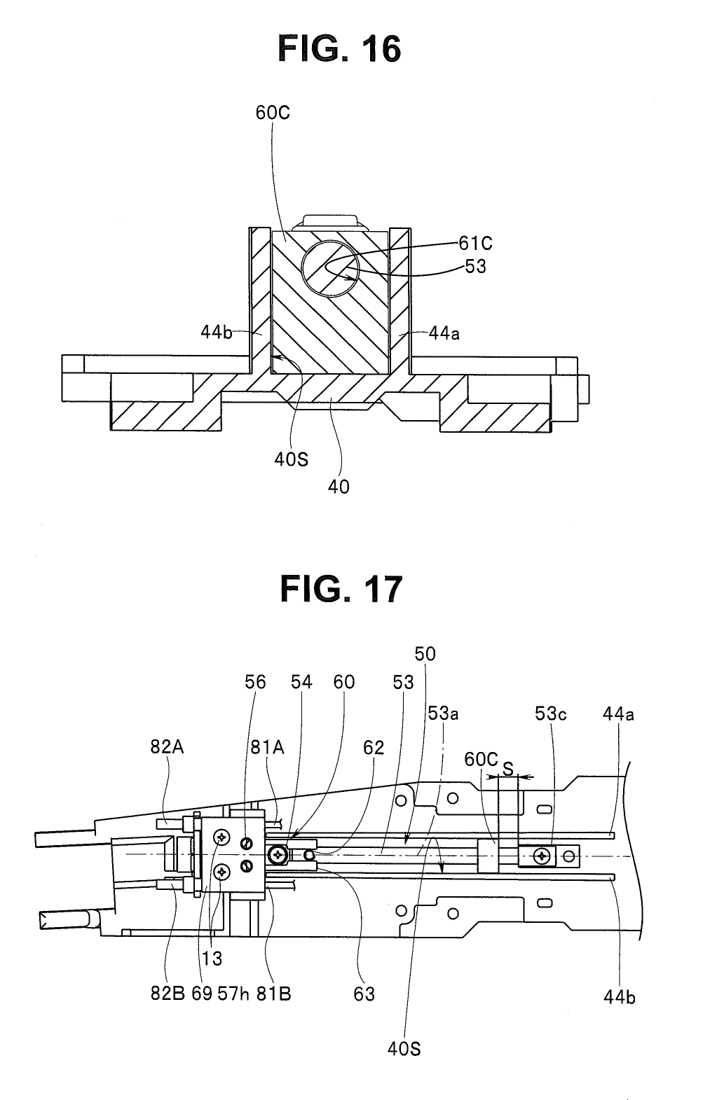

Further, as shown in FIGS. 15 to 17, a second restricting member 60C may be provided on a second end side of the slide member 53. The second restricting member 60C is provided with a restricting hole 61C, which is an axial direction through hole. A diameter dimension of the restricting hole 61C is set to correspond to a predetermined fitting state so that the slide member 53 smoothly moves back and forth without backlash.

In the present embodiment, a distal end side end of the coupling cover 53c of the slide member 53 comes into contact with a proximal end surface of the second restricting member 60C in the inner coil non-fixed state as shown in FIG. 15, and the distal end side end of the coupling cover 53c is separated from the proximal end surface of the second restricting member 60C by the clearance S in the inner coil fixed state as shown in FIG. 17.

Thus, by providing the second restricting member 60C having the restricting hole 61C on the second end side of the slide member 53, it is possible to cause the slide member 53 to be disposed horizontally relative to the surface of the bottom plate 40 with high accuracy.

Note that the present invention is not limited only to the embodiment described above, but various modifications can be performed within a range not departing from the spirit of the invention.

According to the present invention, it is possible to realize an endoscope effectively transmitting an operation force inputted to a slide member via a transmitting member to a functional portion so that a desired function can be certainly obtained.

What is claimed is:

1. An endoscope comprising:
a functional portion having a function of, by rotating around a rotation shaft formed in a first direction, pressing in a second direction from a pressing portion formed on an outer circumferential surface;
a link mechanism attached to the rotation shaft, rotatable around the rotation shaft, and having a through hole formed in the first direction;
a shaft member disposed in the through hole and capable of moving in a cross direction crossing the first and second directions by causing the link mechanism to rotate around the rotation shaft;
a slide member fixed to the shaft member, having a longitudinal axis in the cross direction, and being moved in the cross direction together with the shaft member by an operation force for causing the functional portion to function being inputted; and
a restricting member configured to restrict the slide member so as not to move in a direction different from the cross direction.

2. The endoscope according to claim 1, wherein
the restricting member restricts the slide member so as not to move in the direction different from the cross direction over a movable range of the shaft member moved accompanying movement of the slide member.

3. The endoscope according to claim 1, wherein
the restricting member has a restricting hole in which the slide member is slidably disposed; and
the slide member is disposed in the restricting hole in a predetermined fitting state.

4. The endoscope according to claim 3, wherein
the restricting member has a shaft member moving groove where the shaft member is slidably disposed accompanying movement of the slide member.

5. The endoscope according to claim 4, wherein
a bottom surface of the shaft member moving groove is provided at a position beyond the restricting hole;
the shaft member has a flange configured to restrict the link mechanism so as not to move in an axial direction of the shaft member, at a proximal end portion of a shaft body; and
a separation distance between one surface of the flange and a link member disposing surface is adjustable.

6. The endoscope according to claim 5, wherein
the shaft member has a male screw to be screwed with a female screw provided in a through hole formed in the slide member, at a distal end portion of the shaft body.

7. The endoscope according to claim 1, wherein
the direction different from the cross direction is the first direction.
